(19) United States
(12) Patent Application Publication
(10) Pub. No.: US 2011/0195919 A1
(43) Pub. Date: Aug. 11, 2011

(54) COMBINATION THERAPY FOR ACNE VULGARIS COMPRISING ADMINISTRATION OF ADAPALENE 0.3% GEL AND CLINDAMYCIN/BENZOYL PEROXIDE GEL

(76) Inventors: Lucy Colon, Fort Worth, TX (US); Ronald Gottschalk, North Richmond Hills, TX (US)

(21) Appl. No.: 12/989,128

(22) PCT Filed: Apr. 24, 2009
(86) PCT No.: PCT/EP2009/055010

§ 371 Date: Mar. 9, 2011

Related U.S. Application Data
(60) Provisional application No. 61/047,661, filed on Apr. 24, 2008.

(51) Int. Cl. A61K 31/7056; A61P 17/10

(52) U.S. Cl. 514/24

(57) ABSTRACT

A novel regime or regimen for the treatment of acne related diseases and particularly acne vulgaris includes administering to a patient in need of such treatment a therapeutically effective amount of Differin gel 0.3% (adapalene) in association or combination with a combined clindamycin/benzoyl peroxide gel product such as DUAC®.

This invention relates to a regime or a regimen for inhibiting or treating acne, comprising administering to an individual subject in need of treatment, an effective amount of Adapalene in association or in combination with a combined Clindamycin/Benzoyl Peroxide Gel product such as a DUAC® product.

The burden of acne is significant, more than 50 million Americans experiencing some form of acne.⁵ Acne vulgaris is a common skin disorder that makes up 20% of the visits to a dermatology practice, and affects the majority of the teenage population; approximately 80% of young adults and adolescents. Management of acne is challenging, especially when considering the chronicity of the disease and the variability in response to treatment.

Acne management can be complex, because the disease is multifactorial, involving various etiological features, including follicular hyperkeratinisation, increased sebum production, P. acnes proliferation, and inflammation.

Oral isotretinoin (13-cis-retinoic acid) is currently the only medication that affects all of the major acne pathogenic factors. However, this drug has been associated with multiple serious side effects, the most serious of which is teratogenicity. For inflammatory acne, insight into alternative treatment approaches such as the association of an oral antibiotic and a topical treatment is beneficial to ensure that oral isotretinoin is reserved for the most severe or aggressive cases of the disease. When such combination is foreseen, The Global Alliance to Improve Outcomes in Acne Guidelines recommend early combination therapy of a topical retinoid and an oral antibiotic.⁹

The recent Consensus Recommendations for the Management of Acne (JAAD sup 2003; 49:1), state that effective acne treatment should target as many of its pathogenic factors, as possible.

The recommendations also state that a topical retinoid should be used in the initial treatment of almost all new patients with acne, because they are the most effective anticomedonal agents currently available. Retinoids help disrupt acne pathogenesis by preventing the development of new microcomedones, and some possess both direct and indirect anti-inflammatory activity.

The management of acne often requires combination therapy and a long-term therapeutic strategy. (See, for example, Thiboutot D. New treatments and therapeutic strategies for acne. Arch Family Med 2000; 9: 179-187; Gollnick H, Cunliffe W, Berson D, et al. Management of acne, a report from a Global Alliance to Improve Outcomes in Acne. J Am Acad Dermatol. 2003; 49 (1 suppl):S1-S37).

In addition, Acne vulgaris is a multi-factorial disease characterized by:
Overproduction of sebum,
Microcomedone and comedone formation caused by hyperkeratosis of the follicular epithelium and retention keratosis,
Proliferation of microbes, particularly P. acnes in the sebum, and
Inflammation resulting from the rupture of comedones.

If not appropriately treated, acne may cause serious physical and emotional scarring and can significantly impact the quality of life of those affected by the disease.

The ideal treatment regimen for the disease would take into consideration the underlying pathology for each of these factors. Unfortunately, except for oral isotretinoin, no single product exists that addresses all of the factors.

Therefore, there is a medical need for a topical treatment addressing most of acne causing factors.

The inventors have now demonstrated that Adapalene or their salts, particularly at a concentration of 0.3%, in association or in combination with a combined Clindamycin/Benzoyl Peroxide Gel product such as a DUAC® product provides excellent results. The addition of molecules with retinoid such as an anti-bacterial activity with an antibiotic makes sense not only as association therapy, but also to prevent future lesion development after oral therapy has been discontinued.

The present invention provides also a regime or a regimen for inhibiting or treating acne related diseases, comprising administering to an individual subject in need of treatment an effective amount of Adapalene particularly at a concentration of 0.3%, in association or in combination with a combined Clindamycin/Benzoyl Peroxide Gel product such as a DUAC® product.
Preferentially, Adapalene and combined Clindamycin/Benzoyl Peroxide product are all applied topically once a day. More preferred, Adapalene is topically applied in the evening and combined Clindamycin/Benzoyl Peroxide product is topically applied in the morning. Conversely, Adapalene is topically applied in the morning and combined Clindamycin/Benzoyl Peroxide product is topically applied in the evening.
In a preferred embodiment, Adapalene is at concentration of 0.3% of weight with regards to the total weight of the Adaplene composition. Preferably, Adapalene is a composition in a form of gel or cream. Also preferred, the combined Clindamycin/Benzoyl Peroxide product is a composition in a form of gel.
According to one embodiment, the regimen is for treating moderate to severe acne.
According to another embodiment, the duration of treatment according to the regime or regimen is from 10 to 16 weeks and preferably 12 to 14 weeks and preferably 12 weeks.
According to another embodiment, the invention encompasses a method of treating acne comprising administering to an individual subject in need of treatment, an effective amount of Adapalene in association or in combination with a combined Clindamycin/Benzoyl Peroxide product.
According to another embodiment, the invention encompasses a kit of part comprising a composition comprising an effective amount of Adapalene and a combined Clindamycin/Benzoyl Peroxide product to be used for treating acne, wherein Adapalene is topically applied in the morning and the combined Clindamycin/Benzoyl Peroxide product is topically applied in the evening or conversely.
The present invention provides also the use of adapalene for the preparation of a composition for the treatment of acne in association or in combination with combined Clindamycin/Benzoyl Peroxide Gel product.

By adapalene is meant the compound 6-[3-(1-adamantyl)-4-methoxyphenyl]-2-naphthoic acid.

It is meant by adapalene salts, the salts obtained or obtainable with a pharmaceutical acceptable base, particularly mineral bases such as sodium hydroxide, potassium hydroxide, ammonium hydroxide or organic bases such as lysine, arginine, N-methyl-glutamine.

It is also meant the salts obtained or obtainable with fatty amine such as dioctylamine and stearylamine.

The adapalene concentration used in the composition according to the invention is then between 0.001 and 5% and advantageously between 0.01% and 1% in weight of adapalene with regards to the total weight of the composition, preferably between 0.01% and 0.5%, and preferentially at least equal to 0.03%, more preferentially between 0.1% and 0.4% and particularly preferred at a concentration of 0.1% and at a concentration of 0.3%.

BPO is a well established antimicrobial agent, is more effective than topical antibiotics on P acnes suppression, with no evidence of microorganism resistance.^{2,} Because retinoids do not create selective pressure for resistance, this combination is expected to decrease the incidence of epidermal bacterial resistance relative to an antibiotic.

Acne vulgaris is a chronic, skin disease of the pilosebaceous unit affecting approximately 80% of young adults and adolescents.¹⁻⁵ The management of acne can be challenging due to the variability in response to treatment and the need for long-term therapy. If not appropriately treated, acne may cause serious physical and emotional scarring and can significantly impact the quality of life of those affected by the disease.⁶ Currently, there is a variety of topical and systemic therapies which are recommended for the treatment of acne, including retinoids, antibiotics, benzoyl peroxide, and hormone therapy. Topical gels, such as Differin® Gel 0.3% and Duac®, are an integral part of acne therapy and are considered appropriate first-line therapy, either alone or in combination with other therapies, for all cases of acne with the exception of the most severe.⁶

Adapalene is a synthetic naphthoic acid derivative with retinoid activity, which has been shown to reverse the abnormal follicular desquamation and inflammatory responses involved in the pathogenesis of acne.⁷⁻¹¹ The efficacy of Adapelene Gel has been established in numerous clinical trials as monotherapy¹¹⁻¹⁵ as well as in combination with other topical and oral antibiotics.¹⁶⁻¹⁷

Adapalene is marketed in two formulations, including a gel, and a cream, and is currently available in two concentrations, 0.1% gel and cream as well as 0.3% gel. For this study, a 0.3% concentration gel will be used.

Duac® Gel is a viscous, opaque, white to slightly yellow, aqueous gel containing both clindamycin phosphate (equivalent to clindamycin 1% and benzoyl peroxide 5%). Clinical studies of the use of Duac® Gel have demonstrated a reduction in Acne vulgaris lesions with tolerability within acceptable limits.¹⁸⁻²¹ Use of the topical formulation of Clindamycin results in the absorption of the antibiotic from the skin surface.²²

Duac® Gel has demonstrated clinical efficacy in the treatment of Acne vulgaris through both antibacterial and anti-inflammatory means.¹⁸ Benzoyl peroxide may exert its antibacterial activity by the interaction of oxidized intermediates with elements of bacterial cells. Clindamycin inhibits bacterial protein synthesis by binding to the 50S ribosomal subunits causing inhibition of peptide-bond formation. Benzoyl peroxide decreases inflammatory damage by inhibiting the release of reactive oxygen species from polymorphonuclear leukocytes (PMNs) through the killing of PMNs. Clindamycin suppresses the complement-derived chemotaxis of polymorphonuclear leukocytes in vitro, thereby reducing the potential for inflammation. The use of Clindamycin in combination with Benzoyl Peroxide (Duac® Gel) is designed to decrease bacterial resistance when compared to clindamycin alone and has also been shown to produce a faster and clinically significant enhancement of the efficacy versus using clindamycin alone.¹⁸⁻²¹

A current common practice in the dermatologist's office is to prescribe a combination treatment consisting of a retinoid product, an antibiotic (topical or oral) and benzoyl peroxide.²² The objective of this study is to determine the efficacy and safety of 12 week treatment with a Differin® Gel (0.3%) in combination with Duac® (Clindamycin/Benzoyl Peroxide Gel) in subjects with moderate to severe Acne vulgaris. It is theorized that combining Adapelene 0.3%

severe Acne vulgaris. Although the combined use of these products may result in noticeable skin irritation, it is believed that the benefit of improved therapy for severe acne will far outweigh the risks.

This invention relates to a regime or a regimen for inhibiting or treating acne, comprising administering to an individual subject in need of treatment, an effective amount of Adapalene in association or in combination with a DUAC® product. In one embodiment of the invention, Adapalene is topically applied in the evening and DUAC® product is topically applied in the morning. In particularly preferred embodiment of the invention, Adapalene is at concentration of 0.3% of weight with regards to the total weight of the adaplene composition.

In one embodiment, Adapalene is a composition in a form of gel or cream and DUAC® is a composition in a form of gel.

The regime or regimen of the instant invention is particularly adapted to treat moderate to severe acne.

According to one embodiment of the invention, the treatment with the composition comprising Adapalene in association or combination with a combined Clindamycin/Benzoyl Peroxide Gel product such as a DUAC® product is carried out once a day. Preferably, the composition comprising Adapalene is applied in the evening and the combined Clindamycin/Benzoyl Peroxide Gel product such as a DUAC® product is administered in the morning or conversely.

Another object of the invention is relating to a method for treating a patient afflicted with acne related disease and particularly acne vulgaris comprising administering to a patient in need of treatment, an effective amount of a composition comprising Adapalene in association or combination with a combined Clindamycin/Benzoyl Peroxide Gel product such as a DUAC® product.

Another object of the invention is relating to the use an effective amount of comprising Adapalene to prepare a composition used in association or combination with a combined Clindamycin/Benzoyl Peroxide Gel product such as a DUAC® product for treating a patient afflicted with acne related disease and particularly acne vulgaris comprising administering to a patient in need of treatment.

In a preferred embodiment, the acne related disease is acne vulgaris. In a more preferred embodiment, acne is moderate to severe acne, preferably severe inflammatory acne vulgaris.

Another object of the invention is relating to regime or regimen as mentioned above wherein administering to a individual subject in need of treatment of an effective amount of Adapalene in association or in combination with a combined Clindamycin/Benzoyl Peroxide product is from 10 to 16 weeks and preferably 12 to 14 weeks.

Preferably another object of the invention is relating to regime or regimen as mentioned above, wherein duration treatment is 12 weeks.

Another object of the invention is relating to a method of treating acne comprising administering to an individual subject in need of treatment, an effective amount of Adapalene in association or in combination with a combined Clindamycin/Benzoyl Peroxide product.

Another object of the invention is relating to a kit of part comprising a composition comprising an effective amount of Adapalene and a combined Clindamycin/Benzoyl

Peroxide product to be used for treating acne, wherein Adapalene is topically applied in the morning and the combined Clindamycin/Benzoyl Peroxide product is topically applied in the evening or conversely.

As illustration of the invention, without limiting the scope, it is provided the following example, where the purpose of the clinical study is to show superiority in terms of efficacy of the association of Adapalene compared to Adapalene vehicle with Duac, in the treatment of Subjects with severe acne vulgaris after a 12-week treatment period.

### EXAMPLE

### Clinical Test of Treatment of Severe Acne Vulgaris With a Gel Composition Containing Adapalene 0.3% Gel Associated With Duac

1. Background and Rationale

The purpose of this study is to determine the efficacy and safety of 12 week treatment with Differin® Gel 0.3%, applied in the evening, in combination with Duac® (Clindamycin/Benzoyl Peroxide Gel) applied in the morning, in Subjects with Acne vulgaris.
2.2. Clinical Hypothesis

The objectives of this study are:
To determine efficacy of the combination therapy in terms of percent change from baseline in total acne lesion counts at Week 12/Early Termination
To demonstrate a local tolerance for 12 weeks of treatment with Differin Gel 0.3% in combination with Duac® (Clindamycin/Benzoyl Peroxide Gel).
3. Selection and Disposition of Study Population
3.1. Number of Subjects

A total of approximately 100 subjects will be enrolled among 4 US sites, with each site enrolling approximately 25 subjects.
3.2. Study Population Characteristics

Male or female subjects of any race, between the ages of 12 to 35 years, with any Skin Type (Dry, Normal, Combination, or Oily), and any Fitzpatrick Skin Type (I-VI) with Acne vulgaris, meeting specific inclusion/exclusion criteria.
3.3. Inclusion Criteria
1. Male or female Subjects of any race, age 12 to 35 years inclusive, with facial Acne vulgaris,
2. Subjects with a minimum of 20 inflammatory lesions (papules and pustules) on the face,
3. Subjects with a minimum of 15 and a maximum of 100 non-inflammatory lesions (open comedones and closed comedones) on the face, excluding the nose,
4. Subject has a Global Severity Assessment score at Baseline of 3 or more.
5. Female Subjects of childbearing potential must have a negative urine pregnancy test (UPT). Females of non-childbearing potential, e.g., premenses, post-menopausal (absence of menstrual bleeding for 1 year), hysterectomy, bilateral tubal ligation, or bilateral ovariectomy, are not required to have a UPT at the beginning of the study,
6. Female Subjects of childbearing potential must practice a highly effective method of contraception during the study: oral contraception (must have been on a stable dose for 3 months prior to study entry), IUD, double-barrier method, systemic (injectable or patch) contraception, strict abstinence or partner had a vasectomy,
7. Subjects (and parent/guardian if Subject is under 18 years of age) must be willing and capable of following study instructions to the extent and degree required by the protocol,
8. Subjects 18 years and older must sign the approved Informed Consent Form prior to any study procedures. Subjects under the age of 18 years must sign an assent-to-participate form, and must have a parent or guardian read and sign the Informed Consent Form prior to undergoing any study procedures. The parent or guardian is not required to attend the follow-up visits unless requested,
9. At selected sites, Subjects must be willing to be photographed. If so required, Subjects (and parent/guardian if Subject is under 18 years of age) must be willing to sign a Photography Release Form.
   3.4. Exclusion Criteria
1. Subjects with more than three nodulo-cystic lesions,
2. Female Subjects who are pregnant, nursing or planning a pregnancy during the study,
3. Subjects with a condition or who are in a situation which, in the Investigator's opinion, may put the Subject at risk, may confound the study results, or may interfere with the Subject's participation in the study,
4. Subjects with known allergy to one of the components of the test products,
5. Subjects who have participated in another investigational drug or device research study within 30 days of enrollment,
6. Subjects with a wash-out period for topical treatment on the face less than (see table below):

| Alpha hydroxyl acid products, astringents, | 1 | day |
|---|---|---|
| preparations with alcohol | | |
| Zinc containing drugs | 1 | week |
| Corticosteroids, antibiotics, antiseptics, | 4 | weeks |
| retinoids, other anti-inflammatory drugs, | | |
| or other acne treatments | | |
| Chemical peels | 4 | weeks |

7. Subjects with a wash-out period for systemic treatment less than (see table below):

| Corticosteroids, antibiotics | 4 | weeks |
|---|---|---|
| Any known photosensitizer (such as Tetracyclines, | 4 | weeks |
| Sulfonamides, Thiazides, Phenothiazines, | | |
| Piroxicam and Psoralens) | | |
| Spironolactone | 3 | months |
| Other systemic acne treatments (including | 6 | months |
| isotretinoin) | | |
| Oral contraceptives for acne treatment (e.g. Ortho | 6 | months |
| Tricyclen ® Yasmin ® Alesse ®) unless at a stable | | |
| dose for the last 6 months | | |
| Ciproterone acetate | 6 | months |

8. Subjects with acne conglobata, acne fulminans, secondary acne (chloracne, drug-induced acne, etc.), or severe nodulocystic acne requiring treatment with oral isotretinoin,
9. Subjects with a beard or other facial hair that might interfere with study assessments,
10. Subjects who are at risk in terms of precautions, warnings, and contra-indication (see package insert for Differin Gel, 0.3% and Duac® Gel),
11. Subjects who foresee intensive UV exposure during the study (mountain sports, UV radiation, sunbathing, etc.).
   3.5. Previous and Concomitant Therapies
      3.5.1. Previous Therapies

Information on previous therapies that have been stopped within the six months preceding Baseline that may have an impact on inclusion/exclusion criteria should be recorded on the Previous Therapy for Acne Form of the Case Report Form (CRF). These therapies should also include all birth control treatments and hormone replacement therapy that have been started, stopped, or maintained in the six months prior to study start.
3.5.2. Concomitant Therapies

The use of any concomitant medication, whether it is a prescription or over-the-counter (OTC) treatment, is to be recorded on the Subject's CRF along with the total daily dosage, units and reason the medication was taken. Any therapy used by the Subject during the study, will be considered as a concomitant therapy.

Subjects will be allowed to continue to use cosmetics during their participation in the study as long as the cosmetics are removed using the Cetaphil® Gentle Skin Cleanser provided by the Investigator. The use of personal care agents such as astringents, toners, clarifying lotions etc. will not be allowed.

Every attempt should be made to keep concomitant therapy dosing and regimen constant during the study. Any change to this therapy should be noted on the Concomitant Therapy Form. If applicable, an Adverse Event Form should be completed for any Subject starting a new concomitant therapy.
4. Investigational Plan
4.1. Overall Study Design

This study will be conducted as an open-label, observational study involving Subjects of any race, Skin Type (Dry, Normal, Combination, or Oily) and any Fitzpatrick Skin Type (I-VI) age 12 to 35 years inclusive, with Acne vulgaris, and meeting other specific inclusion/exclusion criteria.

A total of 100 Subjects will be enrolled in 4 sites in the USA. Approximately 25 Subjects will be enrolled from each site.

Subjects will be enrolled at Baseline and treated for 12 weeks with Differin® Gel, 0.3% in combination with Duac® Topical Gel. Subjects will be evaluated at five study visits: Baseline, Week 2, Week 6, Week 8 and Week 12/Early Termination.

Subjects will apply Duac® to the face in the morning and Differin® 0.3% to the face in the evening.
4.2. Discussion of Study Design

Several independent studies of Differin® 0.1% and Duac® Gel have been conducted which suggest that each has comparable efficacy, but Differin® 0.1% is better tolerated.^{11-15, 18-22} However, to date, no previous study has combined Differin® Gel, 0.3% with Duac® Gel.

In an effort to address a current common practice in dermatologist's office to prescribe a combination regimen that includes a retinoid, antibiotic (topical or oral) and/or a benzoyl peroxide product, this study's aim is to determine the efficacy and safety of 12 week treatment with the combined treatment of Differin® Gel 0.3% and Duac® Gel. Moreover, it is an opportunity to explore this treatment regimen in comparison to the recommended treatment with each medication separately.

The objective of this study is to determine the efficacy and safety of 12-week treatment with Differin® Gel 0.3% in combination with Duac® Gel.

Because all the medications are being used for their approved indications once daily (one in the evening and one in the morning), this study is considered a Phase IV study. Each Subject will apply Duac® Gel in the morning and Differin® Gel 0.3% in the evening once daily. Eligible Subjects will be evaluated five times (Baseline, Week 2, Week 6, Week 8, and Week 12). To ensure proper assessment of medication tolerability, a Week 2 study visit will be scheduled. A urine pregnancy test is required at Baseline, Week 2, Week 6, Week 8 and Week 12/Early Termination for all females of childbearing potential.
4.3. Study Flow Chart

| | STUDY VISITS | | | | |
|---|---|---|---|---|---|
| | | | | | Week 12^{a} |
| | | | | | (+/−5 days)/ |
| | | Week 2 | Week 6 | Week 8 | Early |
| PROCEDURES | Baseline | (+/−3 days) | (+/−5 days) | (+/−5 days) | Termination |
| Informed Consent/Assent | X | | | | |
| Demographics/Medical History | X | | | | |
| Previous Therapies^{b} | X | | | | |
| Inclusions/Exclusions Criteria | X | | | | |
| Urine Pregnancy Test^{c} | X | X | X | X | X |
| Global Severity Assessment | X | X | X | X | X |
| Lesion counts^{d} | X | X | X | X | X |
| Local Tolerability^{e} | X^{f} | X | X | X | X |
| Investigator Global Assessment | | | | | X |
| of Improvement | | | | | |
| Investigator Satisfaction Survey | | | | | X |
| Subject Satisfaction Survey | | | | | X |
| Photographs at selected sites | X | X | X | X | X |
| Treatment Dispensed/Tube weight | X | | X | | |
| Treatment Returned/Tube Weight | | X | X | X | X |
| Concomitant Therapy | X | X | X | X | X |
| Adverse Events | X | X | X | X | X |
| Subject Diary/Review instructions | | X | X | X | X |
| Exit form | | | | | X |

^{a}All study procedures should be conducted earlier if Subject discontinues prior to Week 12 and recorded on the appropriate pages of the CRF.
^{b}Therapy that continues after Baseline should be recorded on the Concomitant Therapy Form of the CRF.
^{c}Urine pregnancy testing is mandatory at each visit for all females of childbearing potential. At the Baseline visit, females of childbearing potential should be in their normal menstrual period before starting any application of the study products.
^{d}Inflammatory lesion counts and Non-Inflammatory lesions counts.
^{e}The Investigator must record and grade the severity of the signs and record the assessment of symptoms of local tolerability (erythema, dryness, scaling, and stinging/burning) at each visit. Tolerability, which requires a dose modification or concomitant treatment, should be recorded as an Adverse Event,
^{f}Evaluate local tolerability at Baseline prior to first application of study products at site.

4.4. Study Visit Description and Procedures
   4.4.1. Baseline Visit
1. Review and explain the nature and the constraints of the study to the Subject (and parent or guardian if the Subject is<18 years of age);
2. Have the Subject (and/or parent/guardian) read and sign an IRB approved Informed Consent (and approved assent form if the Subject is<18 years of age). Give a signed copy to each Subject/representative;
3. Question the Subject about demography, medical history, length of time their acne has been present, previous and concomitant therapies. If the Subject requires a medication washout period, the Subject's Baseline evaluation must be conducted after the washout period is completed;
4. Evaluate the Subject according to inclusion and exclusion criteria (see sections 3.3 and 3.4);
5. If applicable, conduct a urine pregnancy test for female Subjects of childbearing potential;
6. Conduct the global severity assessment of the face;
7. Conduct the initial facial inflammatory and non-inflammatory lesion counts;
8. Grade and record the severity of the signs and symptoms related to tolerability (erythema, scaling, dryness, and stinging/burning) prior to the first application of study products;
9. At selected sites, take photographs of the face according to specific photographic procedures (see Section 6.3.1 and Investigator Manual);
10. Assign the Subject number, and dispense one tube of each study medication. Complete the provided drug labels. Record the weight of the tubes, date of dispensation and initials of the person dispensing the medications on the Drug Dispensation/Return Form;
11. Provide verbal and written instructions on how to properly use the study products and how to use the Subject diary to keep a record of study medication application. The first dose of topical medications will be applied by the Subject under the direction of study personnel before leaving the investigational center;
12. Question Subject and record occurrence of Adverse Events;
13. Schedule Week 2 post-Baseline visit approximately 14 days from the Baseline visit.
   4.4.2. Follow-Up Visits (Week 2, Week 6 and Week 8)
1. Conduct the global severity assessment of the face;
2. Conduct the facial inflammatory and non-inflammatory lesion counts;
3. Grade and record the severity of the signs and symptoms related to tolerability (erythema, dryness, scaling, and stinging/burning);
4. At selected sites, take photographs of the face according to specific photographic procedures (see Section 6.3.1 and Investigator Manual);
5. Conduct a urine pregnancy test on all female Subjects of childbearing potential;
6. Record study medication compliance; at the Week 6 visit, collect the study medication, and dispense new tubes of treatment as applicable. Record the weight of the tubes, date of dispensation and initials of the person dispensing the medications on the Drug Dispensation/Return Form;
7. Question the Subject about the occurrence of Adverse Events;
b 8. Inquire as to whether concomitant therapies have been added, stopped, or changed since the Subject's last visit. Document all changes on the Case Report Form;
9. Review the Subject's diary carefully;
10. At the Week 6 visit, dispense enough study products to last for the remaining 6 weeks of the study; Duac® will be dispensed at the Week 6 visit as it has a 60 day expiration date.
11. Schedule the next follow up visit;
12. In case of any premature termination of the study whatever the reason is, conduct all activities required for the Early Termination Visit.
   4.4.3. Week 12 /Early Termination Visit
1. Conduct the global severity assessment of the face;
2. Conduct the facial inflammatory and non-inflammatory lesion counts;
3. Grade and record the severity of the signs and symptoms related to tolerability (erythema, dryness, scaling, and stinging/burning);
4. Conduct the Investigator global assessment of improvement from Baseline.
5. At selected sites; take photographs of the face according to specific photographic procedures (see Section 6.3.1 and Investigator Manual);
6. Conduct a urine pregnancy test on all female Subjects of childbearing potential;
7. Ensure that Subject has returned all tubes of study products. All missing tubes must be explained on the Drug Dispensation/Return Form. Record weight of all tubes on the Drug Dispensation/Return Form;
8. Provide the Subject with the Subject's Satisfaction Survey and review the questionnaire for completion;
9. Ask the Investigator/Evaluator to Complete a Satisfaction Survey and review it for completion;
10. Collect and carefully review the Subject's diary;
11. Question the Subject about the occurrence of adverse events;
12. Inquire as to whether concomitant therapies have been added, stopped, or changed since the Subject's last visit. Document all changes on the Case Report Form;
13. Complete the Exit Form.
4.5. Study Duration and Termination

This study is estimated to have duration of approximately 12 months from time of initial Subject enrollment to the completion of the last Subject. Study duration for each Subject is approximately 12 weeks.

The study may be terminated by the Investigator at his/her investigative center at any time with appropriate notification to Galderma Laboratories L.P. Likewise, Galderma Laboratories L.P. may terminate the clinical study and/or investigative center with appropriate notification.
5. Test Materials

The study materials, Differin® Gel, 0.3%, Duac® Topical Gel, Cetaphil® Gentle Skin Cleanser, Cetaphil® Daily Facial Moisturizer SPF 15, and Urine Pregnancy Tests (First Response™ or equivalent) will be provided by the Sponsor
5.1.4. Instructions for Use and Administration

Differin® Gel, 0.3% and Duac® Topical Gel will be dispensed for each enrolled subject initially for 6 weeks of treatment, then dispensed again, if necessary, at the 6 Week visit for another 6 weeks of treatment. Subjects will treat the face with Duac® once daily in the morning and with Differin® Gel, 0.3% once daily in the evening for 12 weeks. The first dose of study medication will be applied by the Subject under the direction of study personnel before leaving the investigational center at the Baseline visit.

The study medication should be applied to the entire face after washing with the provided Cetaphil® Gentle Skin Cleanser. All Subjects will apply study medication immediately after washing their face with Cetaphil® Gentle Skin Cleanser. Medicated shaving creams should not be used during the study. During the initial interview, subjects will be queried as to whether or not they use shaving cream on their face. If they use shaving cream, the brand will be recorded on the Concomitant Medication Case Report Form.

The treatment administration is further described in the following table.

| Differin ® Gel, 0.3% | | Duac ® Topical Gel |
|---|---|---|
| Concentration | Adapalene 0.3% | 1% Clindamycin |
| | | 5% Benzoyl Peroxide |
| Dose Regimen | Once daily in the | Once daily in the |
| | evening | morning |
| Period of Administration | 12 weeks | 12 weeks |
| Route of Administration | Topically to the face | Topically to the face |

The study products will be returned at each applicable visit to the Investigator. At the end of the study, all used and unused study medication will be returned to the Sponsor.
5.1.5. Other Study Supplies

Cetaphil® Gentle Skin Cleanser, Cetaphil® Daily Facial Moisturizer SPF 15, and Urine Pregnancy Tests (First Response™ or equivalent) will be provided by the Sponsor. Subjects should use the Cetaphil® Gentle Skin Cleanser to wash the affected area prior to applying study medication. Subjects should use the provided Cetaphil® moisturizer as required for the symptomatic relief of skin dryness or irritation. No accountability will be conducted on these products. If the Subject does not want to use the Cetaphil® Gentle Skin Cleanser and Cetaphil® Daily Facial Moisturizer SPF 15, the cleanser and moisturizer they choose to use will be documented on the Concomitant Medication Case Report Form. These products should not contain any ingredients listed in the Prohibited Therapies (section 3.5.2.2).
6. Efficacy and Safety Assessment

Clinical evaluations should be performed by the same Evaluator throughout the study. If it is not possible to use the same Investigator or Evaluator to follow the Subject, then evaluations should overlap (examine the Subject together and discuss findings) for at least one visit.
6.1. Efficacy Assessment
   6.1.1. Efficacy Criteria

The Primary Efficacy Criterion is the percent change from baseline in total lesion count at week 12.

The Secondary Efficacy Criteria will be:
Percent change from baseline in total lesion counts at Week 6
Percent change from baseline in inflammatory lesion counts at Week 6 and 12.
Percent change from baseline in non-inflammatory lesion counts at Week 6 and 12.
Global severity assessment at full scale at Weeks 6 and 12.
Global severity assessment on a dichotomous scale (success or failure) at Weeks 6 and 12.
Evaluator Global assessment of improvement from baseline at Week 12.

All primary and secondary endpoints will be summarized with descriptive statistics for each evaluation time point (Baseline, Week 2, Week 6, Week 8, and Week12).
6.1.2. Efficacy Measurements

The Evaluator will conduct efficacy evaluations at each visit consisting of non-inflammatory lesions and inflammatory lesions count, global severity assessment at full scale, and finally Evaluator global assessment of improvement from Baseline at Week 12. Photographs will be taken at selected investigational centers for documentation of progression of disease and marketing and publication purposes.
6.1.3. Lesion Counts

Each type of lesion will be counted separately and recorded on the appropriate Case Report Form. The Investigator (or the Evaluator) will take the lesion counts from the forehead, left and right cheeks, and chin above the jaw line (excluding the nose). The lesion counts will be electronically added together to obtain a total lesion count. The following are the definitions of the lesions that will be counted.

### Non-Inflammatory Lesions

Open Comedone—A mass of sebaceous material that is impacted behind an open follicular orifice (blackhead).
Closed Comedone—A mass of sebaceous material that is impacted behind a closed follicular orifice (whitehead).

### Inflammatory Lesions

Papules—A small, solid elevation less than 5 mm in diameter.
Pustules—A small, circumscribed elevation of the skin less than 5 mm in diameter, which contains yellow-white exudate.
Nodules/Cysts—A circumscribed, elevated, lesion greater than or equal to 5 mm in diameter.
   6.1.4. Global Severity Assessment

The Evaluator will assess the severity (global grade) of acne at Baseline and at each post-Baseline visit. The global severity assessment will be used to define the acne severity. The Evaluator will evaluate the Subject's acne at each visit performing a static (“snap-shot”) evaluation of acne severity. The Evaluator should make no reference to Baseline or other previous visits when evaluating the Subject's facial acne. This clinical instrument will be dichotomized into clinical success (grades 0 and 1 or a two grade improvement) and failure (grades 2, 3, 4 and 5) at the end of the study by the statistician. The global severity assessment is outlined in the following table:

Investigator's Global Severity Assessment

| | | | |
|---|---|---|---|
| Success | 0 | Clear | Residual hyperpigmentation and |
| | | | erythema may be present. |
| | 1 | Almost Clear | A few scattered comedones and a few |
| | | | (less than five) small papules. |
| Failure | 2 | Mild | Easily recognizable; less than half the |
| | | | face is involved. Many comedones |
| | | | and many papules and pustules. |
| | 3 | Moderate | More than half of the face is involved. |
| | | | Numerous comedones, papules and |
| | | | pustules. |
| | 4 | Severe | Entire face is involved. Covered with |
| | | | comedones, numerous papules and |
| | | | pustules and few nodules and cysts. |
| | 5 | Very Severe | Highly inflammatory acne covering the |
| | | | face; with nodules and cysts present. |

6.1.5. Global Assessment of Improvement

The Evaluator will conduct a Global Assessment of Improvement by comparing Week 12 (or Early Termination) facial skin condition to skin condition at Baseline. Subjects will be evaluated according to the following scale:

Global Assessment of Improvement from Baseline

| | | |
|---|---|---|
| 0 | Clear | All signs and symptoms of disease have resolved |
| | | (100% improvement from Baseline) |
| 1 | Almost Clear | Nearly all signs and symptoms cleared (90% |
| | | improvement from Baseline). Only minimal |
| | | residual signs and symptoms remain |
| 2 | Marked | Majority of the signs and symptoms have |
| | Improvement | resolved (about 75% improvement from Baseline) |
| 3 | Moderate | Significant improvement, but many sighs and |
| | Improvement | symptoms remain (about 50% improvement |
| | | from Baseline) |
| 4 | Minimal | Slight overall improvement, but not clinically |
| | Improvement | significant (about 25% improvement from |
| | | Baseline) |
| 5 | No Change | Overall severity similar from Baseline |
| 6 | Worse | Worse than Baseline |

6.2 Safety Assessment

Safety assessments will be conducted for all Subjects at each visit after enrollment in the study. The required safety assessments are the recorded tolerability assessments (erythema, scaling, dryness, and stinging/burning) and reported adverse events. All clinical medical events, whether observed by the Investigator or reported by the Subject and whether or not thought to be drug-related, will be considered adverse events and recorded on the appropriate Adverse Event Case Report Form (see section 7 for the follow-up of AE).
6.2.1. Tolerability Assessment

Erythema, scaling, dryness, and stinging/burning, will be graded at Baseline and each post-Baseline visit as follows:

Erythema-abnormal redness of the skin.

| | | |
|---|---|---|
| None | 0 | No erythema |
| Mild | 1 | Slight pinkness present |
| Moderate | 2 | Definite redness, easily recognized |
| Severe | 3 | Intense redness |

Scaling-abnormal shedding of the stratum corneum.

| | | |
|---|---|---|
| None | 0 | No scaling |
| Mild | 1 | Barely perceptible shedding, noticeable |
| | | only on light scratching or rubbing |
| Moderate | 2 | Obvious but not profuse shedding |
| Severe | 3 | Heavy scale production |

Dryness-brittle and/or tight sensation.

| None | 0 | No dryness |
|---|---|---|
| Mild | 1 | Slight but definite roughness |
| Moderate | 2 | Moderate roughness |
| Severe | 3 | Marked roughness |
| Stinging/Burning-prickling pain sensation immediately | | |
| after (within 5 minutes) dosing. | | |
| None | 0 | No stinging/burning |
| Mild | 1 | Slight warm, tingling/stinging sensation; |
| | | not really bothersome |
| Moderate | 2 | Definite warm, tingling/stinging sensation |
| | | that is somewhat bothersome |
| Severe | 3 | Hot, tingling/stinging sensation that has |
| | | caused definite discomfort |

Erythema, scaling, and dryness will be evaluated, by the Evaluator. Stinging/burning, within the previous 24 hours, will be recorded by the Evaluator after discussion with the Subject. Tolerability changes, which may require a dose modification or concomitant treatment, should be recorded in the Adverse Event form of the CRF.
6.3. Other Assessments
   6.3.1. Photographs

Subjects at selected sites will be photographed at each visit. These photographs will be used for documentation of progression of disease and marketing and publication purposes The selected investigational sites will be trained by Canfield Scientific Inc. and detailed instructions and documentation of training filed in the Investigator's Manual.
6.3.2. Subject's Satisfaction Survey

At Week 12 or Early Termination, Subjects will complete a satisfaction questionnaire regarding the treatments they have been using in this study. All questions will be answered numerically on a scale of 1-10 with 10 being the best.
1. Overall, how well did you like using the treatment?
2. Were you bothered by the treatment side effects?
3. Would you like to continue using the same treatment?
4. Do you believe that the treatment was effective at treating your acne?
5. Overall, how do you feel about your appearance?
   6.3.3. Investigator's Satisfaction Survey

At Week 12 or Early Termination, Evaluators will complete a satisfaction survey regarding the treatment provided to the Subject during this study. All questions will be answered numerically on a scale of 1-10 with 10 being the best.
1. Overall, did you feel the Subject was assigned an appropriate treatment regimen for his/her condition?
2. Was the Subject bothered by the treatment side effects?
3. How likely are you to continue the Subject's treatment as assigned during this study?
4. Do you believe that the treatment was effective at treating this Subject's acne?

5. Overall, how do you feel about the Subject's appearance?
7. Incidence of Adverse Events

Safety assessment will be conducted for all Subjects at each visit after enrolment in the study. All clinical medical events, whether observed by the Investigator or reported by the Subject and whether or not thought to be drug-related, will be considered adverse events and recorded on the appropriate Adverse Event Case Report Form. Throughout the course of the study, all adverse events will be monitored and reported on an Adverse Event Form without omitting any requested and known information. When adverse events occur, the main concern is the safety of the study Subjects.
7.1. Definitions
   7.1.1. Adverse Events (AE)

An adverse event (AE) can be any unfavorable and/or unintended sign (including an abnormal laboratory finding), symptom, or disease temporally associated with the use of a medicinal (investigational) product, whether or not related to the investigational product.

Thus any new sign, symptom or disease, or clinically significant increase in the intensity of an existing sign, symptom or disease, should be considered as an adverse event.

Notes:
Clinically significant worsening of the disease/condition being evaluated, which occurs during the study, is considered an adverse event.
Any new sign or symptoms suffered by the Subject which appears after accidental or intentional overdose or misuse should also be reported as an adverse event.

Any adverse event, whether or not it is related to the study products, will be reported on the Adverse Event form along with the date of onset, the severity, the relationship with the study product and the outcome.

If the Subject discontinues due to an Adverse Event, the Adverse Event and Exit Forms must be completed.

Most common side effects that may be experienced with the use of Differin® Gel, 0.3% include erythema, scaling, dryness, pruritus, and burning in 10-40% of patients. Pruritus or burning immediately after application also occurs in approximately 20% of patients. The following additional adverse experiences were reported in approximately 1% or less of patients: skin irritation, burning/stinging, erythema, sunburn, and acne flares. These are most commonly seen during the first month of therapy and decrease in frequency and severity thereafter. All adverse effects with use of Differin® Gel 0.3% during clinical trials were reversible upon discontinuation of therapy.

The most frequent adverse reactions reported during clinical trials with Duac® Topical Gel in the treatment of acne, occurring in 5-26% of patients, in descending order included erythema, peeling, dryness and burning. The course of these expected events will be assessed and reported on the tolerability assessments. An entry will be made on the Adverse Event Form for all adverse events.

Diarrhea, bloody diarrhea, and colitis (including pseudomembranous colitis) have been reported with the use of topical and systemic Clindamycin. Studies indicate a toxin(s) produced by Clostridia is one primary cause of antibiotic-associated colitis. The colitis is usually characterized by severe persistent diarrhea and severe abdominal cramps and may be associated with the passage of blood and mucus. If significant diarrhea occurs, the drug should be discontinued. Mild cases of pseudomembranous colitis usually respond to drug discontinuation alone. In moderate to severe cases, consideration should be given to management with fluids and electrolytes, protein supplementation and treatment with an antibacterial drug clinically effective against Clostridium difficile colitis.²³
7.1.2. Serious Adverse Events (SAE)

A serious adverse event is any untoward medical occurrence that at any dose:
results in death,
is life-threatening,
requires inpatient hospitalization or prolongation of existing hospitalization,
results in persistent or significant disability/incapacity, or
results in a congenital anomaly/birth defect.

And also:
Other important medical events that jeopardize the Subject or require intervention to prevent one of the outcomes listed above.
Note: The term “life-threatening” refers to an event in which the Subject was at risk of death at the time of event; it does not refer to an event that hypothetically might have caused death if it was more severe.

Hospitalization solely for the purpose of diagnostic tests, even if related to an adverse event, elective hospitalization for an intervention which was already planned before the inclusion of the Subject in the study, and admission to a day-care facility may not themselves constitute sufficient grounds to be considered as a serious adverse event. Hospitalization is defined as admission to a hospital for a period of greater than 24 hours.

Any pregnancy occurring during clinical trials, where the fetus could have been exposed to the investigational product(s), must be reported in the same manner as a SAE and followed-up until outcome in order to ensure the complete collection of safety data on Galderma Laboratories products.
8. Statistical Methods Planned
8.1. Statistical and Analytical Plans

The main purposes are to estimate efficacy in terms of percent change from baseline in total acne lesion counts at Week 12, to demonstrate a local tolerance for 12 weeks of treatment with Differin® Gel in combination with Duac® Topical Gel, and to show that this combination is an effective treatment for severe cases of acne. For efficacy, both the intent to treat and per protocol analyses will be performed.
8.1.1. Variables to be Analyzed

The following variables will be analyzed:
Demographics: Age, gender, skin type (Dry, Normal, Combination and Oily), Fitzpatrick Skin Type, race and ethnicity.
Efficacy parameters:
   Primary Efficacy Parameter is the percent change from baseline in total lesion count at week 12.
   Secondary Efficacy Parameters are:
      Percent change from baseline in total lesion counts at week 6.
      Percent change from baseline in inflammatory lesion counts at weeks 6 and 12.
      Percent change from baseline in non-inflammatory lesion counts at weeks 6 and 12.
      Global severity assessment at full scale at weeks 6 and 12 (ordinal scale rated from 0 to 5).
      Global severity assessment on a dichotomous scale (success or failure) at weeks 6 and 12.

Global assessment of improvement from baseline at endpoint at week 12 (ordinal scale rated from 0 to 6).
Safety Parameters
   Tolerability assessments
   Incidence of Adverse Events
Other Parameters are:
   Investigator Satisfaction Survey
   Subject Satisfaction Survey
   Subject compliance
   8.1.2. Populations Analyzed, Evaluability and Limitation/Evaluation of Bias

The following populations will be analyzed:
1. The intent-to-treat population will include all subjects enrolled and dispensed study medication. This will be the primary population for efficacy analyses. Last observation carried forward (LOCF) will be used to impute missing values from the last non-missing value forward for the efficacy variables only. If no post-Baseline data is available, the Baseline value will be carried forward. ITT efficacy variables will also be presented in separate tables as observed values only for consistency with other Galderma Differin® 0.3% studies. No analysis will be performed on the observed data.
2. The safety population will include all subjects enrolled and with documentation of at least one application of either study product.
3. The per-protocol population will be a subset of the intent-to-treat population. The per-protocol population will be analyzed using observed data only.

Subjects will be eligible for the per-protocol population if they complete all required visits and study evaluations without noteworthy study protocol violations (e.g., any subject or investigator activity that could have possibly interfered with the administration of the study treatments or evaluations of treatment efficacy and safety). A subject will be included in the per-protocol analyses if all of the following criteria are met:
A subject who meets the inclusion/exclusion criteria.
The subject has not taken or applied any interfering concomitant medications.
The subject has completed all required visits and study evaluations within the visit window indicated in the flow chart.
The subject has been compliant with the dosing regimen (e.g. subject must apply study medication at 80% to 120% of the expected applications of both treatments. Dosing compliance for subjects who prematurely discontinue use of study medication during the treatment phase due to lack of efficacy or adverse events will be based on the number of days the subjects participated in the treatment phase of the study).

Subjects who prematurely discontinue from the study due to a treatment related adverse event will be included in the per-protocol analysis regardless of whether or not they are dosing compliant at the time of discontinuation. Subjects who prematurely discontinue from study due to lack of efficacy will be included in the per-protocol population. However, dosing compliance for these subjects will be computed based on number of days in the treatment phase of the study. These subjects must be 80-120% compliant with the dosing regimen to be included in the per-protocol population.

Preliminary assessment of dosing compliance will be established prior to database lock based on a 12 week dosing schedule. Subsequent to database lock, dosing compliance will then be reviewed for the subjects enrolled and any subject who was not dosing compliant will then be excluded from the per-protocol population (with the exception of those subjects who discontinue due to adverse event or lack of efficacy as described in the preceding paragraph). Documentation and sign-off of changes to the per-protocol population following database lock will be obtained prior to any analyses being performed.
8.1.4. Statistical Analyses

All efficacy variables will be summarized at each visit with mean, median, SD, range or with frequencies. The primary analysis is the ITT analysis of percent change from Baseline in total lesion count at week 12 (LOCF). This analysis will also be conducted based on the PP population to confirm the results.

A Wilks-Shapiro test will be used to determine the normality of percent change from baseline in total lesion counts at week 12. A skewed distribution is expected. Thus, a non-parametric distribution free method will be used to estimate the 95% confidence interval for median percent change from baseline at week 12 (SAS Proc Univariate, confidence limits for percentiles, Hahn and Meeker, 1991). If the data is found to be normally distributed, then the 95% confidence interval for the mean percent change from baseline will be calculated based on parametric methods.

Similar methods will be used to calculate 95% confidence intervals for percent change from baseline for total lesion counts at week 6, and for non-inflammatory and inflammatory lesion counts at each of weeks 6 and 12.

Percent change from baseline at 12 weeks will be compared to percent change from baseline at 6 weeks for each of total lesion counts, non-inflammatory lesion counts, and inflammatory lesion counts using the signed rank test or a paired t-test, depending on the findings from the Wilks-Shapiro test for normality.

Global severity assessment will be summarized with frequency tables at each visit on the full ordinal scale and on the dichotomous scale, where success is defined as ‘clear or almost clear’. The exact binomial test will be used to determine whether week 6 and week 12 success rates are

significantly different from zero. McNemar's test or the sign test (dependent on minimum expected cell size) will be used to compare week 6 scores to week 12 scores on the dichotomous scale. The Wilcoxon signed rank test will be used to compare week 6 and week 12 scores on the full ordinal scale to baseline as well as to compare week 6 to week 12 scores on the full ordinal scale.

Global assessment of improvement from baseline will be summarized with frequency tables at each visit on the full ordinal scale. No formal statistical analyses will be used to analyze global assessment of improvement.

Safety, tolerability, subject satisfaction, investigator satisfaction, and compliance data will be summarized using descriptive statistics as described in 8.1.3. No formal statistical analyses will be conducted for these variables.

No adjustments of p-values for multiplicity will be made. No interim analyses are planned. SAS® software will be used for all data analyses and tabulations, unless otherwise stated.
8.2. Sample Size Determination

On hundred (100) subjects will be enrolled with an expectation that no more than 10% will drop out during the study. Thus, the result will be at least 90 subjects completing the study.

Expected percent mean percent change from baseline in total lesion counts for Differin 0.3% plus Duac combination is 51.67%. This number was derived from first determining that the ratio of mean percent change from baseline total lesion counts for Differin 0.1% alone to Differin 0.1% plus Clindamycin from the MORE Differin 0.1% study was 0.892. Since the mean percent change from baseline total lesion counts from the integrated analysis Differin 0.3% studies 18081 and 18060 was 46.09, the ratio of 0.892 was applied to determine that the expected mean percent change from baseline for Diferin 0.3% plus Duac is 51.67%. The assumed standard deviation was 34.736, taken from the integrated analysis of study 18081 and 18060.

Given 90 subjects, and an expected mean percent change from baseline in total lesion counts of 51.67%, and an assumed standard deviation of 34.736, we will be able to estimate a two-sided 95% confidence interval for percent change from baseline lesion counts of approximate width of 15%, with 44.17 and 59.17 percent change from baseline as the limits of the confidence interval.

### REFERENCES

1. Kraining K K, Odland G F, eds. Prevalence, morbidity, and cost of dermatologic diseases. J Invest Dermatol. 1979; 73:395-513.
2. Cunliffe W J, Gould D J. Prevalence of facial acne vulgaris in late adolescence and in adults. BMJ. 1979; 1:1109-1110.
3. Usatine R P, Quan M A, Strick R. Acne vulgaris: a treatment update. Hosp Pract. 1998; 33:111-127.
4. Leyden J J. New understandings of the pathogenesis of acne. J Am Acad Dermatol. 1995; 32:S15-S25.
5. Pawin H, Beylot C, Chivot M, et al. Physiopathology of acne vulgaris: recent data, new understanding of the treatments. Eur J Dermatol. 2004; 14:4-12.
6. Gollnick H, Cunliffe W, Berson D, et al. Management of acne. A report from a

Global Alliance to Improve Outcomes in Acne. J Am Acad Dermatol. 2003; 49 (1 suppl):S1-S37.
7. Gollnick H, Schramm M. Topical drug treatment in acne. Dermatology. 1998; 196:119-125.
8. Brogden R N, Goa K L. Adapalene: a review of its pharmacological properties and clinical potential in the management of mild to moderate acne. Drugs. 1997; 53:511-519.
9. Michel S, Jomard A, Demarchez M. Pharmacology of adapalene. Br J Dermatol. 1998; 139 (suppl 52):3-7.
10. Vega B, Jomard A, Michel S. Regulation of human monocyte toll-like receptor 2 (TLR2) expression by adapalene. J Eur Acad Dermatol Venereol 2002; 16 (suppl 1):123-124 [Abstract].
11. Cunliffe W J, Caputo R, Dreno B, Föström L, Heenen M, Orfanos C E, et al. Clinical efficacy and safety comparison of adapalene gel and tretinoin gel in the treatment of acne vulgaris: Europe and US multicenter trials. J Am Acad Dermatol. 1997; 36:S126-S134.
12. Shalita A, Weiss J S, Chalker D K, Ellis C N, Greenspan A, Katz I H, et al. A comparison of the efficacy and safety of adapalene gel 0.1% and tretinoin gel 0.025% in the treatment of acne vulgaris: a multicenter trial. J Am Acad Dermatol. 1996; 34:482-485.
13. Grosshans E, Marks R, Mascaro J M, Torras H, Meynadier J, Alirezai M, et al. Evaluation of clinical efficacy and safety of adapalene 0.1% gel versus tretinoin 0.025% gel in the treatment of acne vulgaris, with particular reference to the onset of action and impact on quality of life. Br J Dermatol. 1998; 139:26-33.
14. Thiboutot D, Gold M H Jarratt M T, et al. Randomized controlled trial of the tolerability, safety, and efficacy of adapalene gel 0.1% and tretinoin microsphere gel 0.1% for the treatment of acne vulgaris. Cutis. 2001; 68:10-19.
15. Cunliffe W J, Poncet M, Loesche C, Verschoore M. A comparison of the efficacy and tolerability of adapalene 0.1% gel versus tretinoin 0.025% gel in patients with acne vulgaris: a meta-analysis of five randomized trials. Br J Dermatol. 1998; 139:48-56.
16. Wolf J E Jr, Kaplan D, Kraus S J, et al. Efficacy and tolerability of combined topical treatment of acne vulgaris with adapalene and clindamycin: A multicenter, randomized, investigator-blinded study. J Am Acad Dermatol. 2003; 49:S211-S217.
17. Cunliffe W J, Meynadier J, Alirezai M, et al. Is combined oral and topical therapy better than oral therapy alone in patients with moderate to moderately severe acne vulgaris? A comparison of the efficacy and safety of lymecycline plus adapalene gel 0.1%, versus lymecycline plus gel vehicle. J Am Acad Dermatol. 2003; 49:5218-5226.
18. Warner G T, Plosker G L. Clindamycin/benzoyl peroxide gel: a review of its use in the management of acne. Am J Clin Dermatol. 2002; 3(5):349-60
19. Comparison of the efficacy and safety of a combination topical gel formulation of benzoyl peroxide and clindamycin with benzoyl peroxide, clindamycin and vehicle gel in the treatments of acne vulgaris. Am J Clin Dermatol. 2001; 2 (1):33-39.
20. Treatment of acne with a combination clindamycin/benzoyl peroxide gel compared with clindamycin gel, benzoyl peroxide gel and vehicle gel: combined results of two double-blind investigations. J Am Acad Dermatol. 1997; 37(4):590-595
21. A combination benzoyl peroxide and clindamycin topical gel compared with benzoyl peroxide, clindamycin phosphate, and vehicle in the treatment of acne vulgaris. Cutis. 2001; February (67):165-169.
22. Benefits of Combination Therapy for Acne. A report by Stiefel Laboratories, Inc. Business Briefing: North American Pharmacotherapy 2004:Issue 2.
23. Center for Drug Evaluation and Research Application Number 50-741 Medical

Reviews: Study 156 (A multicenter Double Blind Comparison of the Efficacy and Safety of Clindoxyl Gel, Benzoyl Peroxide Gel, and Clindamycin Gel in the Once Daily Treatment of Acne Vulgaris for 11 Weeks—page 27 and Study 158 (A Multicenter, Double-Blind Comparison of the Efficacy and safety of Clindoxyl Gel, Benzoyl Peroxide Gel, Clindamycin Gel, and Vehicle Gel in the Once Daily

Treatment of Acne Vulgaris for 11 Weeks)—page 40.
24. Lucky A. W., Barber B. L., Girman C. J., Williams J., Ratterman J., Waldstreicher J.
A multirater validation study to assess the reliability of acne lesion counting. J Am Acad Dermatol 1996 Oct; 35 (4):559-65.
25. Hahn, G. J., Meeker, W. Q. (1991), Statistical Intervals: A Guide for Practitioners, New York: John Wiley & Sons, Inc.

1-13. (canceled)

14. A regime or a regimen for inhibiting or treating acne, comprising administering to an individual subject in need of such treatment, for such period of time as required to elicit the desired effect, a therapeutically effective amount of adapalene in association or in combination with a an integrated clindamycin/benzoyl peroxide medicament.

15. The regime or regimen as defined by claim 14, comprising individually topically applying each of said adapalene and integrated clindamycin/benzoyl peroxide medicament once a day.

16. The regime or regimen as defined by claim 14, comprising topically applying the adapalene in the evening and topically applying the integrated clindamycin/benzoyl peroxide medicament in the morning.

17. The regime or regimen as defined by claim 14, comprising topically applying the adapalene in the morning and topically applying the integrated clindamycin/benzoyl peroxide medicament in the evening.

18. The regime or regimen as defined by claim 14, wherein the adapalene is at concentration of 0.3% of weight with respect to the total weight of the composition comprised thereof.

19. The regime or regimen as defined by claim 14, wherein the adapalene comprises a gel or cream thereof.

20. The regime or regimen as defined by claim 14, wherein the integrated clindamycin/benzoyl peroxide medicament comprises a gel.

21. The regime or regimen as defined by claim 14, comprising the treatment of moderate to severe acne.

22. The regime or regimen as defined by claim 14, comprising administering to a individual subject in need of such treatment, a therapeutically effective amount of adapalene in association or in combination with an integrated clindamycin/benzoyl peroxide medicament for from 10 to 16 weeks.

23. The regime or a regimen as defined by claim 22, comprising treatment for from 12 to 14 weeks.

24. A regime or regimen for the treatment of acne vulgaris, comprising administering to an individual subject in need of such treatment, for such period of time as required to elicit the desired effect, a therapeutically effective amount of adapalene in association or in combination with an integrated clindamycin/benzoyl peroxide medicament.

25. A kit useful for the treatment of acne, separately comprising a therapeutically effective amount of adapalene and separately comprising an integrated clindamycin/benzoyl peroxide medicament, the adapalene being indicated for topically application in the morning and the integrated clindamycin/benzoyl peroxide medicament being indicated for topical application in the evening or conversely.
